(19) United States
(12) Patent Application Publication
(10) Pub. No.: US 2004/0152783 A1
(43) Pub. Date: Aug. 5, 2004

(54) Controlled absorption of admixed thyroid hormone formulations

(76) Inventors: Lawrence Peter Olon, Bristol, TN (US); James Scott Moncrief, Christiansburg, VA (US); Nancy Johnston Boerth, Blacksburg, VA (US); Thomas Piccariello, Blacksburg, VA (US)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/701,173

(22) Filed: Nov. 5, 2003

Related U.S. Application Data
(60) Provisional application No. 60/423,595, filed on Nov. 5, 2002.

(51) Int. Cl. A61K 31/198

(52) U.S. Cl. 514/567

(57) ABSTRACT

The invention relates to admixtures of the thyroid hormones and derivatives thereof. These admixtures, particularly L-thyroxine (T4) and liothyronine (T3), produce steady state or near steady state serum levels of T3. When admixed in the appropriate ratio of T4 to T3, blood levels of T3 hormone are maintained at a steady state for an extended period of time independent of the need for control release excipients. The change in the kinetics of T3 absorption is controlled by the addition of T4 to T3.

### FIELD OF INVENTION

The invention relates to admixtures of the thyroid hormones and derivatives thereof. These admixtures, particularly L-thyroxine (T4) and liothyronine (T3), produce steady state or near steady state serum levels of T3. When admixed in the appropriate ratio of T4 to T3, blood levels of T3 hormone are maintained at a steady state for an extended period of time independent of the need for control release excipients. The change in the kinetics of T3 absorption is controlled by the addition of T4 to T3. Orally administered admixtures of T4 and T3 are therapeutically valuable when formulated at an appropriate ratio.

### BACKGROUND

The thyroid gland secretes two hormones: L-tetraiodothyronine (thyroxine, T4) and L-triiodothyronine (triiodothyronine, T3, also called liothyronine). In tissues thyroxine (T4) serves as a precursor for triiodothyronine. Most triiodothyronine in the body comes from enzymatic conversion of thyroxine by tissues rather than from secretion by the thyroid gland. Many patients suffer from reduced or impaired thyroid function which causes deficiencies in T4 and T3. Reduced thyroid function can be due to a number of reasons, including for example thyroidectomy necessitated by thyroid cancer.

Thyroid hormones T4 and T3 play a crucial role in metabolic homeostasis and affect the function of virtually every organ system. In healthy individuals, serum concentrations of the thyroid hormones are controlled by a classic negative-feedback system involving the thyroid gland, the pituitary gland, the hypothalamus and peripheral tissues, such as the liver. In response to the thyroid-stimulating hormone (TSH; also known as thyrotropin) produced by the pituitary, the thyroid gland normally releases a daily production rate of thyroxine of about 100 μg. The daily production rate of triiodothyronine is about 30 μg, of which about 20 percent is produced by the thyroid gland and 80 percent by deiodination of thyroxine in extrathyroidal tissues, particularly the liver. Synthesis and release of TSH by the pituitary is stimulated by thyroid-releasing hormone (TRH) a tripepetide produced by the hypothalamus in response to changes in metabolism caused by low levels of the thyroid hormones. Thyroid hormone replacement therapy normally includes administering T4 to the patient at levels intended to approximate the T4 production levels of a normal thyroid gland. It is generally believed that since T4 is the natural precursor of triiodothyronine (T3), that adequate supplementation of T4 will result in adequate levels of T3 by way of the body's normal conversion of T4 to T3.

Hypothyroidism is a common condition. It has been reported in the United States Federal Register that Hypothyroidism has a prevalence of 0.5 percent to 1.3 percent in adults. In people over 60, the prevalence of primary hypothyroidism increases to 2.7 percent in men and 7.1 percent in women. Because congenital hypothyroidism may result in irreversible mental retardation, which can be avoided with early diagnosis and treatment, newborn screening for this disorder is mandatory in North America, Europe, and Japan. Thyroid disorders are common and include hyper- and hypothyroidism. Hypothyroidism is typically characterized by an elevated level of TSH, but varies widely in its clinical presentation. Furthermore, while some patients present with obvious clinical symptoms, others require the use of biochemical tests to determine the status of thyroid function. As a result, hypothyroidism is generally considered to be under diagnosed.

In recent years, a number of hypothyroid syndromes with subtle presentations have been identified. Subclinical hypothyroidism refers to a condition marked by normal levels of T4 and T3 with elevated TSH. “Euthyroid sick syndrome” and “low T3 syndrome” refer to a condition where low serum levels of T3 are present but normal TSH and T4 levels are observed. These conditions have been associated with a number of nonthyroidal illnesses including congestive heart failure, clinical depression, mood disorders, and obesity. Whether thyroid hormone replacement therapy is efficacious in the treatment of such disorders remains to be established.

Thyroid hormone drugs are natural or synthetic preparations containing tetraiodothyronine (T4, levothyroxine) sodium or triiodothyronine (T3, liothyronine) sodium or both. T4 and T3 are produced in the human thyroid gland by the iodination and coupling of the amino acid tyrosine. T4 contains four iodine atoms and is formed by the coupling of two molecules of diiodotyrosine (DIT). T3 contains three atoms of iodine and is formed by the coupling of one molecule of DIT with one molecule of monoiodotyrosine (MIT). Both hormones are stored in the thyroid colloid as thyroglobulin. Thyroid hormone preparations belong to two categories: (1) natural hormonal preparations derived from animal thyroid, and (2) synthetic preparations. Natural preparations include desiccated thyroid and thyroglobulin. Desiccated thyroid is derived from domesticated animals that are used for food by man (either beef or hog thyroid), and thyroglobulin is derived from thyroid glands of the hog.

Synthetic forms for both T4 and T3 thyroid hormone are available from a number of producers. For example, liothyronine sodium (T3) tablets is available under the trademark Cytomel. Levothyroxine sodium (T4) is available under tradenames Eltroxin; Levothroid; Levoxine; Levoxyl; Synthroid.

Thyroid hormone replacement therapy can be a chronic, lifetime endeavor. The dosage is established for each patient individually. Generally, the initial dose is small. The amount is increased gradually until clinical evaluation and laboratory tests indicate that an optimal response has been achieved. The dose required to maintain this response is then continued. The age and general physical condition of the patient and the severity and duration of hypothyroid symptoms determine the initial dosage and the rate at which the dosage may be increased to the eventual maintenance level. It has been reported that the dosage increase should be very gradual in patients with myxedema or cardiovascular disease to prevent precipitation of angina, myocardial infarction, or stroke.

It is important that thyroid hormone treatment have the correct dosage. Both under treatment and over treatment can have deleterious health impacts. In the case of under treatment, a sub-optimal response and hypothyroidism could result. Under treatment has also been reported to be a potential factor in decreased cardiac contractility and increased risk of coronary artery disease. Conversely, over treatment may result in toxic manifestations of hyperthyroidism such as cardiac pain, palpitations, or cardiac arrhythmia's, particularly in patients with coronary heart disease.

Hyperthyroidism is a known risk factor for osteoporosis. Several studies suggest that subclinical hyperthyroidism in premenopausal women receiving thyroid hormone drugs for replacement or suppressive therapy is associated with bone loss. To minimize the risk of osteoporosis, it is preferable that the dose be kept to the lowest effective dose.

T3 is metabolically active via binding nuclear thyroid hormone receptors and modulating transcription of specific genes. T4 is far less active in the regulation of transcription and is generally considered a prohormone. The metabolic effects of T4 result from the conversion of T4 to T3 by deiodinase enzymes in peripheral tissues, and at the subcellular level once T4 enters a target cell. As noted previously, the T3 in circulation is largely the result of T4 to T3 conversion in the liver.

The use of the active hormone, T3, as replacement therapy in hypothyroid conditions has met with limited success primarily because occasionally rapid increases in serum concentrations, or “spiking” levels, of this hormone in the serum occur, which could prove dangerous to patients whose cardiac status is compromised. For this reason, therapy with the prohormone, T4, has become the treatment of choice in hypothyroidism since, to be active, it first must be converted to T3, in vivo, a process which eliminates the potential for spiking T3 serum levels and any serious sequela. However, recent studies of T4 suggest that a general decline in a patient's ability to convert T4 to T3 is associated with aging, and also has been observed where stress or concurrent disease is present. Additionally, a deficiency in the T4 to T3 conversion capacity of particular organs or organ systems may exist. Given the problems associated with the use of either T3 or T4 as thyroid hormone replacement as herein identified, there is a need for an efficient, effective, low-cost and readily available mechanism for the delivery of thyroid hormones and derivatives thereof. Further, there is a need for compositions and methods to treat hypothyroid conditions and control the absorption of T3 in vivo.

Various methods and formulation are disclosed in the art to deliver thyroxine medications along with some which discuss sustained release and/or stabilize thyroxine medications. Examples of such disclosures include PCT/US99/04311, U.S. Pat. No. 6,458,842, U.S. Pat. No. 4,870,106, PCT/EP95/00322, PCT/US99/12979, U.S. Pat. No. 6,190,696, U.S. Pat. No. 6,056,975, U.S. Pat. No. 5,955,105, U.S. Pat. No. 5,856,359, EP 0742714B1, U.S. Pat. No. 5,752,254, and U.S. Pat. No. 5,955,105. These disclosures typically disclose the use of T3 alone, T4 alone or T3 in combination with T4 wherein less T3 is less in amount as compared to T4. Other patents describe ratios of T4:T3 above a 1:1 ratio, but do not provide guidance directed to, optimal ranges or the pharmacological effects of various ranges, which might effect dosage amounts used to achieve proper blood levels. See for instance, U.S. Pat. No. 5,324,522, U.S. Pat. No. 3,477,954, EP 1077681B1, U.S. Pat. No. 3,577,535, U.S. Pat. No. 3,917,832, U.S. Pat. No. 5,958,979 and U.S. 2002/0193440. Other patents discuss means for measuring serum levels of T3 and T4, such as PCT/US01/23593.

Each of above is hereby incorporated by reference in its entirety to the extent not in conflict with the current application, particularly for the uses, conditions and formulations for delivery. For instance, the compositions of the present invention may also be used to cataracts according the procedures of Shihao or formulated for sustained release, but utilizing the ratios of T4:T3 described herein.

This invention relates to pharmaceutical compositions and methods of their use in treating patients having reduced thyroid gland function. In particular, the invention relates to compositions comprising and methods of using such a composition to treat a variety of symptoms associated with decreased thyroid function.

Because of the risks associated with over treatment or under treatment with levothyroxine sodium, the need for thyroid hormone products that are consistent in potency and bioavailability remains an issue. A need therefore remains for an improved therapeutic regimen which in addition to treating hypothyroid patients also addresses remaining symptoms such as reduced mental performance, reduced cognitive function, and mental depression.

### BRIEF DESCRIPTION OF THE DRAWINGS

FIG. 1. Delta TT3 serum concentration curves of T4:T3 (13.5:1) vs. T3 sodium;

FIG. 2. Delta TT3 serum concentration curves of T4:T3 (9:1) vs. T3 sodium;

FIG. 3. Delta TT3 serum concentration curves of T4:T3 (4:1) vs. T3 sodium;

FIG. 4. Delta TT3 serum concentration curves of T4:T3 (1:1) vs. T3 sodium;

FIG. 5. Delta TT3 serum concentration curves of T4:T3 (0.4:1) vs. T3 sodium;

FIG. 6. Delta TT3 serum concentration curves of T4:T3 (0.1:1) vs. T3 sodium.

### DESCRIPTION OF THE INVENTION

Due to the exceedingly low doses of T3 that are therapeutically valuable it has been difficult to establish a reliable sustained release formulation using control release agents. Control release agents may include dissolution methods, microspheres, press tablet formulations, coatings, micropumps, etc. Thus alternative mechanisms for establishing slow release or absorption of T3 would be particularly valuable in therapeutic applications of T3 administration.

The invention embodies specific ratios of T4 to T3 in establishing steady state or near steady state levels of T3 over an extended period of time, preferably through oral administration. Preferably the steady state absorption rates approach zero order pharmacokinetics. When T4 and T3 are admixed in particular ratios a total T3 (TT3) concentration curve can be established that is identical to a sustained absorption profile. In general when higher ratios of T4 to T3 (approximately greater than 4:1) are given, crossover of the TT3 curve can be established when the curve is compared to that of an equivalent dose of T3. Additionally, when lower ratios of T4 to T3 (approximately less than 1:1) are given, crossover of the TT3 curve can again be established when the curve is compared to that of an equivalent dose of T3. Without wishing to be restricted to this theory, it appears the presence of T4 in the T4:T3 admixture delays the absorption of T3. Therefore, the peak of change (delta of serum concentration) in the TT3 curve of a given dose of T3 can be decreased by adding T4 to the dose. At the proper ratio approximately equal bioavailability can be established due to the complex nature of T4 and T3 absorption and metabolism.

At higher ratios (approximately greater than 4:1) when the correct amount of T4 is added crossover may occur despite the decrease in peak TT3 concentrations. This may be due, in part, to T4 to T3 conversion following absorption of T4. T4 to T3 conversion is temporal due to the need for enzymatic conversion by endogenous deiodinase activity, thus there is a delay in the appearance of T3 derived from T4 in circulation. At the proper range of ratios enough T4 is gradually converted to T3 to compensate for the decrease in delta AUC of TT3 concentration due to the presence of T4. Approximately equal bioavailability with steady state levels of TT3 can be established when the proper high ratio is found.

At low ratios (approximately less than 1:1) when the correct amount of T4 is added crossover may occur despite the decrease in peak TT3 concentrations. This may be due to T4 delaying the absorption of T3. At the proper range of ratios T3 absorption is temporally controlled by the presence of T4 such that steady state or near steady state levels of T3 are maintained over a period of time that is useful for therapeutic delivery of T3.

At ratios between about 1:1 and about 4:1 the presence of T4 results in sustained absorption TT3 concentration curve; however, the delta AUC of these doses is not equal to an equivalent dose of T3. Therefore, only doses with high enough or low enough T4:T3 ratios are valuable for obtaining sustained release of T3 with approximately equal bioavailability as compared to the T3 equivalent dose.

One embodiment of the invention provides a pharmaceutical composition comprising levothyroxine (T4) or a derivative thereof and liothyronine (T3) or a derivative thereof in an admixture, wherein said admixture has a ratio which creates in vivo liothyronine serum levels at steady state or near steady state for an extended period of time. In a preferred embodiment the admixture ratio of T4:T3 is less than 1:1, more preferably less than 0.95:1, more preferably less than 0.90:1, more preferably less than 0.85:1, more preferably less than 0.80:1, more preferably less than 0.75:1, more preferably less than 0.70:1, more preferably less than 0.65:1, more preferably less than 0.60:1, more preferably less than 0.55:1, more preferably less than 0.40:1, more preferably less than 0.35:1, more preferably less than 0.30:1, more preferably less than 0.25:1, more preferably less than 0.20:1, more preferably less than 0.15:1, more preferably less than 0.10:1, more preferably less than 0.05:1. In one embodiment, the most preferred range is between 0.4:1 and 0.1:1. It should be recognized that the methods preferably utilize any of the recited ratios of the invention.

In another embodiment, the admixture ratio of T4:T3 is above 4:1, more preferably about 5:1, more preferably above 6: 1, more preferably above 7:1, more preferably above 8:1, more preferably above 9:1, more preferably above 10:1. While ranges may be used, for instance at levels above 20:1, above 30:1, above 40:1, or above 50:1, the preferred range is between about 4:1 and about 10:1, and more preferably between about 5:1 and about 9:1. It should be recognized that the methods preferably utilize any of the recited ratios of the invention.

While compositions and methods of the invention may be delivered through various means as described in the specification a preferably the compositions are designed for oral administration, i.e. tablets, capsules, caplets, solutions, and suspensions.

One aspect of the invention provides compositions and methods which result in a peak total liothyronine serum concentration that is less than the peak total liothyronine concentration resulting from an equivalent dose of liothyronine alone. Another aspect of the invention provides method and compositions which result in liothyronine bioavailability equal to or nearly equal to an equivalent amount of liothyronine administered alone. In yet another aspect, the compositions and methods prevent or reduce the spiking of liothyronine serum levels.

The methods and compositions of the invention are useful in the treatment of various disorders and conditions in humans and animals. Preferably, the methods and compositions are used to treat hypothyroidism, subclinical hypothyroidism, hyperthyroidism, depression, obesity, congestive heart failure, and various mood disorders.

For each of the recited embodiments levothyroxine and liothyronine derivatives are separately selected from salt forms, esterified derivatives, amide linked derivatives, or combinations thereof. Likewise, an alternative embodiment either or both of levothyroxine and liothyronine is linked to one or more amino acid residues as described in the art. In another embodiment either or both of levothyroxine and liothyronine may be is combined with at least one other control release agent.

Another method of the invention creates a the steady state of liothyronine concentration in vivo through the administration of an admixture of liothyronine or derivatives thereof and levothyroxine or derivatives thereof wherein said steady state is dependent on a particular ratio of levothyroxine to liothyronine and does not require other control release agents.

In another embodiment, the methods of the invention are supplemented or combined with a control release agent in the formulation to further improve steady-steady state or time release of the T3 or T4. In a preferred embodiment the T3 or T4 is covalently linked to one or more amino acid residues.

The methods of the invention also provide for improved steady state pharmacokinetics, preferably with increased bioavailability. In particular, methods of the invention achieve bioequivalence at a lower ratio of total daily drug delivered compared to when said admixture is used alone. Another embodiment provides a method of creating a steady state liothyronine concentration in vivo comprising administering an admixture of liothyronine or derivatives thereof and levothyroxine or derivatives thereof wherein said steady state is modulated by a ratio of levothyroxine to liothyronine and does not require other control release agents. Another embodiment provides a method of increasing the bioavailability of T4 and/or T3 comprising administering an admixture of liothyronine or derivatives thereof and levothyroxine or derivatives in a ratio of T4:T3 which is above 4:1 or below 1:1. While another embodiment provides a method for treating a condition requiring the modulation of T3 and/or T4 comprising administering levothyroxine (T4) or a derivative thereof and liothyronine (T3) or a derivative thereof in a ratio of T4:T3 which is above 4:1 or below 1:1. In a preferred embodiment the condition is hypothyroidism, subclinical hypothyroidism, hyperthyroidism, depression, obesity, congestive heart failure, or a mood disorder.

One aspect of this technology is that the desired endpoint for both sustained release and sustained absorption results in modulated absorption. Existing sustained and extended release pharmaceutical formulations depend on specialized polymer matrices that control the dissolution rate of drugs due to swelling and porosity properties of the polymers. One aspect of the present invention embodies the use of ligand competition as part of the mechanism for sustained absorption of T3. The absorption of T3 is slowed by the presence of T4 which competes for a shared receptor on the surface of the intestinal tract; thus the term sustained absorption is used to describe the pharmacokinetics of the T4 plus T3 admixtures. This is in contrast to the usual methods of achieving steady state or near steady state levels of drugs by controlling the release of the drugs as they descend the intestinal tract.

It is also possible for the dosage form to combine any forms of release known to persons of ordinary skill in the art. These include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is known in the art.

Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, creams, topicals, inhalants, aerosol inhalants, patches, particle inhalants, implants, ingestibles, injectables (including subcutaneous, intramuscular, intravenous, and intradermal), infusions, health bars, confections, animal feeds, cereals, yogurts, cereal coatings, foods, nutritive foods, functional foods and combinations thereof. Most preferably the compositions and methods of the invention utilize a form suitable for oral administration.

Formulations of the present invention suitable for oral administration can be presented as discrete units, such as capsules or tablets. These oral formulations also can comprise a solution or a suspension in an aqueous liquid or a non-aqueous liquid. The formulation can be an emulsion, such as an oil-in-water liquid emulsion or a water-in-oil liquid emulsion.

Soft gel or soft gelatin capsules may be prepared, for example by dispersing the formulation in an appropriate vehicle (vegetable oils are commonly used) to form a high viscosity mixture. This mixture is then encapsulated with a gelatin based film using technology and machinery known to those in the soft gel industry.

Chewable tablets, for example may be prepared by mixing the formulations with excipients designed to form a relatively soft, flavored, tablet dosage form that is intended to be chewed rather than swallowed. Conventional tablet machinery and procedures, that is both direct compression and granulation, i.e., or slugging, before compression, can be utilized. Those individuals involved in pharmaceutical solid dosage form production are versed in the processes and the machinery used as the chewable dosage form is a very common dosage form in the pharmaceutical industry.

Film coated tablets, for example may be prepared by coating tablets using techniques such as rotating pan coating methods or air suspension methods to deposit a contiguous film layer on a tablet.

Compressed tablets, for example may be prepared by mixing the formulation with excipients intended to add binding qualities to disintegration qualities. The mixture is either directly compressed or granulated then compressed using methods and machinery known to those in the industry. The resultant compressed tablet dosage units are then packaged according to market need, i.e., unit dose, rolls, bulk bottles, blister packs, etc.

The invention also contemplates the use of biologically-acceptable carriers which may be prepared from a wide range of materials. Without being limited thereto, such materials include diluents, binders and adhesives, lubricants, plasticizers, disintegrants, colorants, bulking substances, flavorings, sweeteners and miscellaneous materials such as buffers and adsorbents in order to prepare a particular medicated composition.

Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives, such as whey, starches, and derivatives, as well as other conventional binders known to persons skilled in the art. Exemplary non-limiting solvents are water, ethanol, isopropyl alcohol, methylene chloride or mixtures and combinations thereof. Exemplary non-limiting bulking substances include sugar, lactose, gelatin, starch, and silicon dioxide.

The plasticizers used in the dissolution modifying system are preferably previously dissolved in an organic solvent and added in solution form. Preferred plasticizers may be selected from the group consisting of diethyl phthalate, diethyl sebacate, triethyl citrate, cronotic acid, propylene glycol, butyl phthalate, dibutyl sebacate, castor oil and mixtures thereof, without limitation.

Formulations suitable for topical administration to the skin can be presented as ointments, creams and gels. In formulations suitable for nasal administration, the carrier is a liquid, such as those used in a conventional nasal spray or nasal drops.

Formulations suitable for parenteral administration include aqueous and non-aqueous sterile injection solutions which optionally can contain antioxidants, buffers, bacteriostats and solutes which render the formulation isotonic with the blood of the intended recipient; and aqueous and non-aqueous sterile suspensions which can include suspending agents and thickening agents. The formulations can be presented in unit-dose or multi-dose containers.

It should be understood that in addition to the ingredients particularly mentioned above, the formulations of this invention can include other suitable agents such as flavoring agents, preservatives and antioxidants. Such antioxidants would be food acceptable and could include vitamin E, carotene, BHT or other antioxidants known to those of skill in the art.

Compositions of the invention may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24 hour period. The doses may be uneven doses with regard to one another or with regard to the individual components at different administration times.

Likewise, the compositions of the invention may be provided in a blister pack or other such pharmaceutical package. Further, the compositions of the present inventive subject matter may further include or be accompanied by indicia allowing individuals to identify the compositions as products for the various disorders (i.e. hypothyroidism, depression, obesity etc.). The indicia may further additionally include an indication of the above specified time periods for administering the compositions. For example the indicia may be time indicia indicating a specific or general time of day for administration of the composition, or the indicia may be a day indicia indicating a day of the week for administration of the composition. The blister pack or other combination package may also include a second pharmaceutical product.

In order to facilitate a more complete understanding of the invention, Examples are provided below. However, the scope of the invention is not limited to specific embodiments disclosed in these Examples, which are for purposes of illustration only.

### EXAMPLES

### In Vivo Performance of T3 Sodium Salt vs. T4 Sodium Salt Mixed with T3 Sodium Salt at Various Ratios

The kinetics of T3 absorption from T3 sodium and various admixtures of T4 sodium mixed with T3 sodium were studied in Sprague-Dawley rats (˜300 g) and are further described below in Examples 1 through 6. Defined μg/kg doses were delivered as oral solutions in 0.5% sodium bicarbonate buffer. Rats were dosed immediately following 0 hour serum collection. Serum was collected from rats at two, four, six, nine, 12 and 24 hours after dosing. Total serum T3 concentrations were determined by ELISA using a commercially available kit (Total Triiodothyronine (total T3) ELISA KIT, product #1700, ALPHA DIAGNOSTIC™, San Antonio, Tex.).

### Example 1

Example 1 illustrates crossover of the delta serum TT3 serum concentration curve of a 162 μg/kg T4 sodium plus 12 μg/kg of T3 sodium dose (13.5:1 weight ratio) vs. a 12 pg/kg dose of T3 sodium.

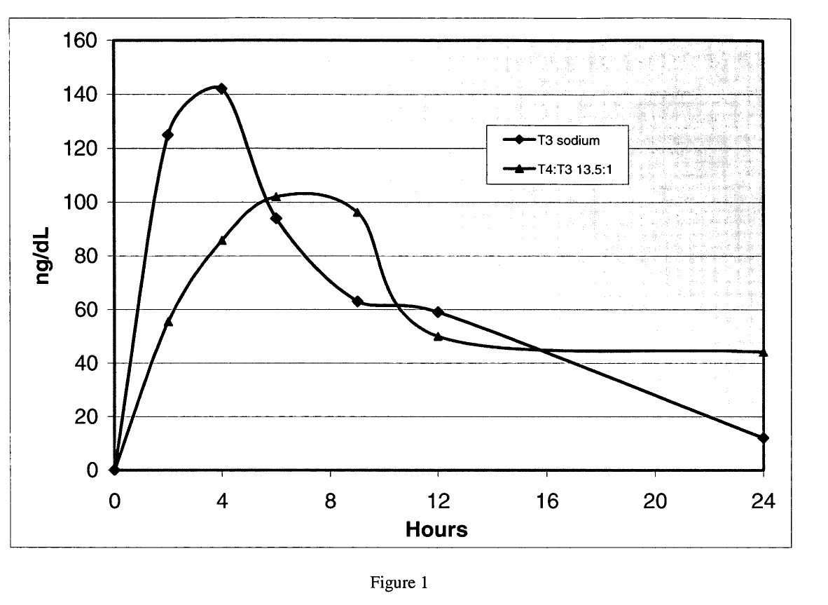

Crossover of the curves occurred at approximately six hours. The delta AUC of the T4 plus T3 admixture compared to the T3 sodium dose was approximately equal (1,469 ng/dL·hr and 1,471 ng/dL·hr, respectively). (See FIG. 1).

### Example 2

Example 2 illustrates crossover of the serum TT3 serum concentration curve of a 54 μg/kg T4 sodium plus 6 μg/kg of T3 sodium dose (9:1 weight ratio) vs. a 6 μg/kg dose of T3 sodium.

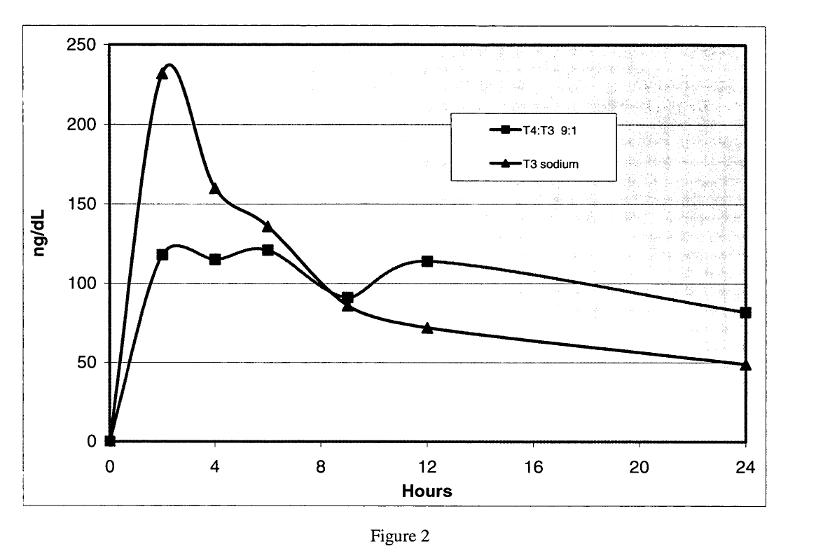

Crossover of the curves occurred at approximately nine hours. The delta AUC of the T4 plus T3 admixture compared to the T3 sodium dose was approximately equal (2,212 ng/dL·hr and 2,384 ng/dL·hr, respectively) (See FIG. 2).

### Example 3

Example 3 illustrates crossover of the serum total T3 serum concentration curve of a 16 μg/kg T4 sodium plus 4 μg/kg of T3 sodium dose (4:1 weight ratio) vs. a 4 μg/kg dose of T3 sodium.

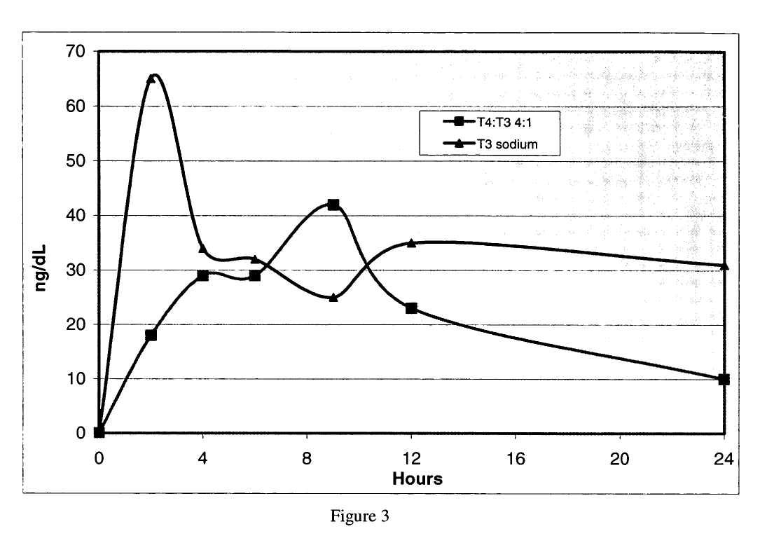

Crossover of the curves occurred at approximately six hours. The delta AUC of the T4 plus T3 admixture, however, was only 44 percent of the T3 sodium dose AUC (527 ng/dL·hr and 1,193 ng/dL·hr, respectively). (See FIG. 3).

### Example 4

Example 4 illustrates the lack of crossover of the serum total T3 serum concentration curve of a 4 μg/kg T4 sodium plus 4 μg/kg of T3 sodium dose (1:1 weight ratio) vs. a 4 μg/kg dose of T3 sodium.

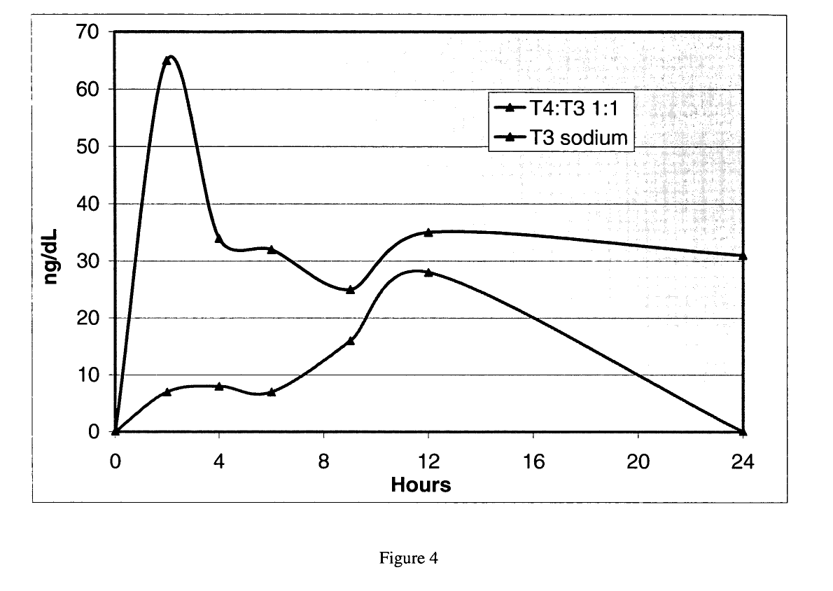

Although absorption of T3 was delayed and decreased, crossover of the curves did not occur. Further the delta AUC of the T4 plus T3 admixture was only 25 percent of the T3 sodium dose AUC (293 ng/dL·hr and 1,193 ng/dL·hr, respectively). (See FIG. 4).

### Example 5

Example 5 illustrates crossover of the serum total T3 serum concentration curve of a 6 μg/kg T4 sodium plus 15 μg/kg of T3 sodium dose (0.4:1 weight ratio) vs. a 15 μg/kg dose of T3 sodium.

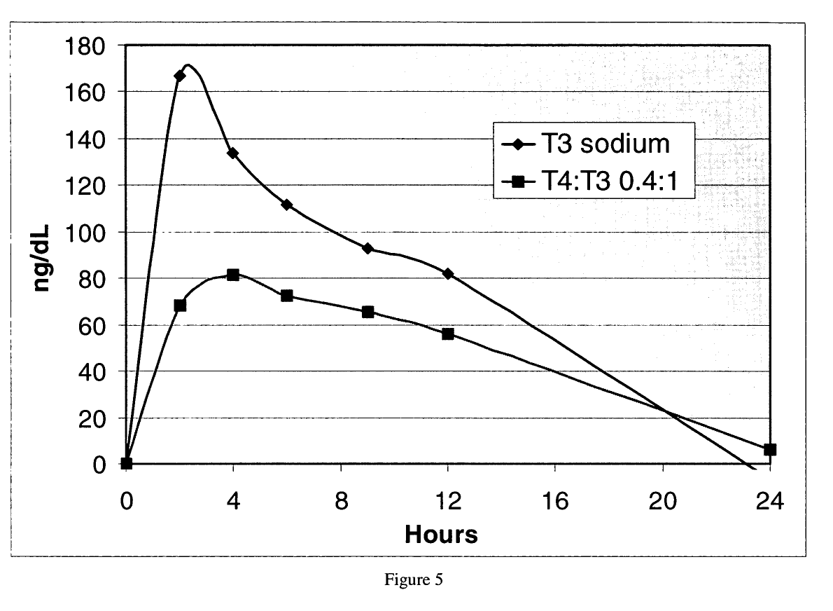

Crossover of the TT3 delta curves occurred at approximately 20 hours. The delta AUC of the T4 plus T3 admixture, however, was only 65 percent of the T3 sodium dose AUC (1,132 ng/dL·hr and 1,732 ng/dL·hr, respectively). (See FIG. 5).

### Example 6

Example 6 illustrates crossover of the serum total T3 serum concentration curve of a 1.5 μg/kg T4 sodium plus 15 μg/kg of T3 sodium dose (0.1:1 weight ratio) vs. a 15 μg/kg dose of T3 sodium.

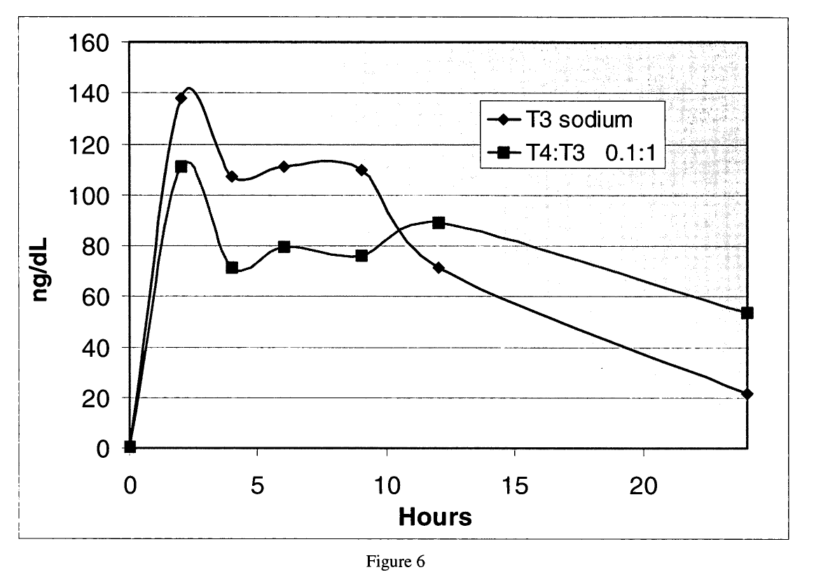

Crossover of the curves occurred at approximately 12 hours. The delta AUC of the T4 plus T3 admixture was approximately equal to that of the T3 sodium dose (1,784 ng/dL·hr and 1,762 ng/dL·hr, respectively). (See FIG. 6).

The in vivo performance of T4 sodium plus T3 sodium at various ratios vs. an equivalent T3 sodium dose is summarized in Table 1.

TABLE 1
Pharmacokinetic Parameters of Orally Administered T4
sodium plus T3 sodium at Various Ratios vs. a T3 sodium
Dose Equivalent to the T3 sodium in the admixture.

| | | | | Peak of | | | | | | | |
|---|---|---|---|---|---|---|---|---|---|---|---|
| | | | T4:T3 | Delta | Percent | | Percent | | Percent | AUC* | Percent |
| | | | Weight | Curve | T3 | Deltamax | T3 | Tₘₐₓ | T3 | (0-24) | T3 |
| Example | Sample | Dose | Ratio | (ng/dL) | sodium | (ng/dL) | sodium | (hours) | sodium | (ng/dLh) | sodium |
| 1 | T3 sodium | 12 | NA | 142 | 100 | 156 | 100 | 5.2 | 100 | 1,469 | 100 |
| | T4 sodium plus | 162 | 13.5:1 | 102 | 72 | 127 | 81 | 9.6 | 185 | 1,471 | 100 |
| | T3 sodium | 12 | | | | | | | | | |
| 2 | T3 sodium | 6 | NA | 232 | 100 | 232 | 100 | 2 | 100 | 2,212 | 100 |
| | T4 sodium plus | 54 | 9:1 | 121 | 52 | 142 | 61 | 10.5 | 525 | 2,384 | 108 |
| | T3 sodium | 6 | | | | | | | | | |
| 3 | T3 sodium | 4 | NA | 65 | 100 | 69 | 100 | 2.4 | 100 | 1,193 | 100 |
| | T4 sodium plus | 16 | 4:1 | 42 | 65 | 54 | 78 | 6 | 250 | 527 | 44 |
| | T3 sodium | 4 | | | | | | | | | |
| 4 | T3 sodium | 4 | NA | 65 | 100 | 69 | 100 | 2.4 | 100 | 1,193 | 100 |
| | T4 sodium plus | 4 | 1:1 | 28 | 43 | 37 | 54 | 7.8 | 325 | 293 | 25 |
| | T3 sodium | 4 | | | | | | | | | |
| 5 | T3 sodium | 15 | NA | 167 | 100 | 170 | 100 | 4.6 | 100 | 1,732 | 100 |
| | T4 sodium plus | 6 | 0.4:1 | 81 | 49 | 115 | 68 | 7.8 | 165 | 1,132 | 65 |
| | T3 sodium | 15 | | | | | | | | | |
| 6 | T3 sodium | 15 | NA | 138 | 100 | 146 | 100 | 5.6 | 100 | 1,762 | 100 |
| | T4 sodium plus | 1.5 | 0.1:1 | 111 | 80 | 124 | 85 | 6.8 | 121 | 1,784 | 101 |
| | T3 sodium | | | | | | | | | | |

*Determined from the mean delta TT3 curve above the 0 hour starting value of y.

Examples 1 and 2 illustrate that when admixtures of high ratios of T4 to T3 (for example, 13.5:1 and 9:1) were administered orally crossover of the delta TT3 serum concentration curves occurred. The peak of the delta TT3 serum concentration curve and the TT3 deltₘₐₓ, were substantially decreased, while Tₘₐₓ was increased. The AUCs of the high ratio admixtures were approximately equal to those of the equivalent T3 sodium doses. Steady state levels of T3 along with approximately equal AUC were afforded by these high T4 to T3 ratio compositions. In the case of high ratios the steady state levels of T3 may be due to a combination of sustained absorption of T3 and gradual conversion of T4 to T3 by deiodinase activity.

Examples 3 and 4 illustrate that when admixtures of medium range ratios of T4 to T3 or example, 4:1 or 1:1) were administered orally there is a flattening of both the curve and peak delta TT3 serum concentration, a substantial TT3 deltaₘₐₓ decrease, and an increase in Tₘₐₓ.) The AUCs of the medium range ratio admixtures, however, were considerably less than those of the equivalent T3 sodium doses. Thus sustained absorption of T3 was afforded by medium range T4 to T3 ratio compositions, however, bioequivalence was not established. The lack of bioequivalence may be due to contribution of T4 to T3 conversion, since the level of T4 in the admixtures is lower. Particularly when compared to high ratios of T4:T3 or low ratios of T4:T3.

Examples 5 and 6 illustrates that when admixtures of low ratios (for example 0.4:1 or 0.1:1) of T4 to T3 were administered orally crossover of the delta TT3 serum concentration curves occurred. The peak of the delta TT3 serum concentration curve and the TT3 deltₘₐₓ were substantially decreased, while Tₘₐₓ was increased. The AUC of the low ratio admixture was approximately equal to that of the equivalent T3 sodium doses. Thus sustained absorption with approximately equal AUC of T3 was afforded by this low T4 to T3 ratio compositions. The increased bioavailability may be due to establishing a low level of T4 in the T4 plus T3 admixture to allow delay in T3 absorption while still allowing an equivalent amount of T3 to be absorbed over the 24 hour period.

Collectively, the results illustrate that an oral dose ratio (or ratios) of T4 to T3 can be determined that will result in steady state levels of T3 in circulation over an extended period of time with approximately equal bioavailability compared to an equivalent oral dose of T3. Useful ratios were empirically determined for rats. Using a similar approach, therapeutically useful ratios could be determined for humans.

### Hypothetical Example 1

A clinical study might include 100 patients suffering from hypothyroidism which is broken into two study groups. Group I will receive a combination of T4 and T3 in a ratio less than 1. Group II will receive hormone replace according to current medical practice (T4 and T3 at a ratio more than 1) for a period of 10 weeks.

Patients will be evaluated at onset, week 1, week 3, week 5 and week 10 through measurements of serum thyrotropin and thyroid hormone. Expected results should demonstrate that Group I (low ratio) showed controlled levels of T3 and T4 along with fewer side effects associated with peaks and valleys resulting from current pharmaceutical administration when compared to Group II.

In the alternative, a similar study could be designed utilizing one or more of the following measurements to demonstrate the efficacy of the invention at treating related disorders. Non-limiting parameters might include cholesterol, triglycerides, blood pressure and psychological tests for cognitive function (e.g. a Digit Symbol Test, a Digit Span Test of the Wechsler Adult Intelligence Scale, and/or a Visual Scanning Test) and psychological state.

Having described the preferred embodiments of the invention, it will be recognized by those skilled in the art that insubstantial differences in dosage amounts and order of administration are possible without departing from the scope of the following claims.

1. A pharmaceutical composition comprising levothyroxine (T4) or a derivative thereof and liothyronine (T3) or a derivative thereof in an admixture, wherein said admixture of T4 to T3 has a ratio which creates in vivo liothyronine serum levels at steady state or near steady state for an extended period of time.

2. A pharmaceutical composition comprising levothyroxine (T4) or a derivative thereof and liothyronine (T3) or a derivative thereof in an admixture wherein said admixture ratio of T4 to T3 is less than 1.

3. A pharmaceutical composition comprising levothyroxine (T4) or a derivative thereof and liothyronine (T3) or a derivative thereof in an admixture, wherein said admixture ratio of T4 to T3 is above 4.

4. The pharmaceutical composition of claim 2 wherein said admixture ratio is below 0.75:1.

5. The pharmaceutical composition of claim 2 wherein said admixture ratio is below 0.5:1.

6. The pharmaceutical composition of claim 2 that delivers a peak total liothyronine serum concentration that is less than the peak total liothyronine concentration resulting from an equivalent dose of liothyronine alone.

7. The pharmaceutical composition of claim 2 that results in bioavailability of liothyronine equal to or nearly equal to an equivalent amount of liothyronine administered alone.

8. The pharmaceutical composition of claim 2 that prevents or reduces spiking of liothyronine serum levels.

9. The pharmaceutical composition of claim 2 wherein levothyroxine and liothyronine derivatives are separately selected from salt forms, esterified derivatives, amide linked derivatives, or combinations thereof.

10. The pharmaceutical composition of claim 2, wherein said admixture is covalently linked to one or more amino acid residues.

11. The pharmaceutical composition of claim 2, wherein said admixture is combined with at least one other control release agent.

12. The pharmaceutical composition of claims 2 in a form suitable for oral administration.

13. The pharmaceutical composition of claim 2 in a form suitable for nasal administration.

14. The pharmaceutical composition of claim 2 in a form suitable as a skin patch.

15. A method of creating a steady state liothyronine concentration in vivo comprising administering an admixture of liothyronine or derivatives thereof and levothyroxine or derivatives thereof wherein said steady state is modulated by a ratio of levothyroxine to liothyronine and does not require other control release agents.

16. The method of claim 15 wherein the steady state or near steady state level of liothyronine concentration is supplemented with a control release agent in the formulation.

17. The method of claim 15 wherein said admixture is covalently linked to one or more amino acid residues.

18. A method of increasing the bioavailability of T4 and/or T3 comprising administering an admixture of liothyronine or derivatives thereof and levothyroxine or derivatives in a ratio of T4:T3 which is above 4:1 or below 1:1.

19. A method for treating a condition requiring the modulation of T3 and/or T4 comprising administering levothyroxine (T4) or a derivative thereof and liothyronine (T3) or a derivative thereof in a ratio of T4:T3 which is above 4:1 or below 1:1.

20. The method of claim 19, wherein said admixture has a ratio of less than 1:1 and greater than 0.05:1.

21. The method of claim 19, wherein said admixture is administered orally or through a skin patch.

22. The method of claim 19, wherein said condition is hypothyroidism, subclinical hypothyroidism, hyperthyroidism, depression, obesity, congestive heart failure, or a mood disorder.
